(19) United States
(12) Patent Application Publication
(10) Pub. No.: US 2014/0378815 A1
(43) Pub. Date: Dec. 25, 2014

(54) MAGNETOENCEPHALOGRAPHY SOURCE IMAGING

(71) Applicant: THE REGENTS OF THE UNIVERSITY OF CALIFORNIA, Oakland, CA (US)

(72) Inventors: Ming-Xiong Huang, San Diego, CA (US); Roland R. Lee, San Diego, CA (US)

(73) Assignee: THE REGENTS OF THE UNIVERSITY OF CALIFORNIA, Oakland, CA (US)

(21) Appl. No.: 14/118,886

(22) PCT Filed: May 24, 2012
(86) PCT No.: PCT/US2012/039478

§ 371 Date: Sep. 15, 2014

Related U.S. Application Data
(60) Provisional application No. 61/489,667, filed on May 24, 2011.

(51) Int. Cl. A61B 5/04; A61B 5/055; G01R 33/48; A61B 5/00

(52) U.S. Cl. CPC A61B 5/04008; A61B 5/4076; A61B 5/055; G01R 33/4806; G01R 33/4808
USPC 600/409

(57) ABSTRACT

Techniques, devices and systems are disclosed for magnetoencephalography (MEG) source imaging. In one aspect, a method includes selecting signal data associated with one or more frequency bands from a spectrum of the signal data in the frequency domain, in which the signal data represents magnetic signals emitted by a brain of a subject and detected by a plurality of sensors outside the brain, defining locations of sources within the brain that generate the magnetic signals, in which the number of locations of the sources is selected to be greater than the number of sensors, and generating a source value of signal power based on the selected signal data corresponding to a respective location of the locations at the one or more frequencies.

### PRIORITY CLAIM

This patent document claims the priority of U.S. provisional application No. 61/489,667 entitled “MAGNETOENCEPHALOGRAPHY SOURCE IMAGING” filed on May 24, 2011, which is incorporated by reference as part of this document.

### STATEMENT REGARDING FEDERALLY SPONSORED RESEARCH OR DEVELOPMENT

This invention was made with government support under grants NEUC-044-065 and NURC-022-10F awarded by the US Department of Veterans Affairs. The government has certain rights in the invention.

### TECHNICAL FIELD

This patent document relates to imaging technologies.

### BACKGROUND

Axonal injury is a leading factor in neuronal injuries such as mild traumatic brain injury (TBI), early multiple sclerosis (MS), early Alzheimer's Disease/dementia (AD), among other disorders. In addition, abnormal functional connectivity exists in these neuronal disorders as well as others, such as post-traumatic stress disorder (PTSD). Neuroimaging tools have been used for diagnosing neurological and psychiatric disorders, e.g., including TBI, PTSD, AD, autism, MS, and schizophrenia. For example, existing neuroimaging techniques can include X-radiation (X-ray), X-ray computed tomography (CT), magnetic resonance imaging (MRI), and diffusion tensor imaging (DTI). Many of these techniques mainly focus on detecting blood product, calcification, and edema, but are less sensitive to axonal injuries and abnormal functional connectivity in the brain. For example, X-ray, CT, and MRI can have poor diagnostic rates to these neurological and psychiatric disorders. For example, less than 10% of mild TBI patients have shown positive findings in X-ray, CT, and MRI. While some techniques such as diffusion tensor imaging (DTI) have shown better sensitivity than X-ray, CT, and MRI neuroimaging in detecting neuronal injuries (e.g., DTI has been shown to produce a positive finding rate ˜20-30% for mild TBI), the vast majority of neuronal injury are left undiagnosed using these neuroimaging techniques.

### SUMMARY

The disclosed technology includes techniques, devices, and systems for solving inverse problems including signal source imaging by employing a frequency-domain vector-based spatio-temporal analysis using L1-minumum norm solution (VESTAL).

In one aspect of the disclosed technology, a method for magnetoencephalography source imaging includes selecting signal data associated with one or more frequency bands from a spectrum of the signal data in the frequency domain, in which the signal data represents magnetic signals emitted by a brain of a subject and detected by a plurality of sensors outside the brain, defining locations of sources within the brain that generate the magnetic signals, in which the number of locations of the sources is selected to be greater than the number of sensors, and generating a source value of signal power based on the selected signal data corresponding to a respective location of the locations at the one or more frequencies.

In another aspect, a method for magnetoencephalography source imaging includes determining a covariance matrix based on MEG signal data in the time domain, in which the MEG signal data represents magnetic signals emitted by a brain of a subject and detected by a plurality of sensors outside the brain, defining locations of sources within the brain that generate the magnetic signals, in which the number of locations of the sources is selected to be greater than the number of sensors, and generating a source value of signal power for each of the locations by fitting the covariance matrix.

In another aspect, an magnetoencephalography source imaging system includes an MEG data acquisition system adapted to acquire magnetic signal data emitted by a brain of a subject that are detected by a plurality of sensors outside the brain, and a data processing unit that receives the magnetic signal data from the MEG data acquisition system, in which the data processing unit includes a mechanism that converts the acquired magnetic signal data from a time domain format into a spectrum of the magnetic signal data in the frequency domain, a mechanism that selects signal data associated with one or more frequency bands from a spectrum of the magnetic signal data in the frequency domain, the frequency bands including one or more frequencies, and a mechanism that generates a source value of signal power based on the selected signal data corresponding to a location within the brain of a source that generates the magnetic signals, in which the source values are generated for the one or more frequencies.

In another aspect, a method for source imaging includes selecting signal data associated with one or more frequency bands from a spectrum of the signal data in the frequency domain, in which the signal data is detected by a plurality of sensors oriented about a structure and the frequency bands include one or more frequencies, defining locations of sources within the structure, in which the number of locations of the sources is selected to be greater than the number of sensors, and generating a source value of signal power based on the selected signal data corresponding to a respective location of the locations for the one or more frequencies.

The subject matter described in this specification potentially can provide one or more of the following advantages. For example, an MEG method using the exemplary frequency-domain VESTAL technology can provide source images with high spatial and temporal resolutions and can detect neuronal injuries and abnormal neuronal networks not visible with other neuroimaging techniques. The disclosed VESTAL techniques in MEG source imaging applications can be implemented in an automated fashion, e.g., without pre-selection of time epochs. These exemplary techniques can be operator independent, e.g., without initial estimations on the number of sources or their locations. The disclosed VESTAL techniques in MEG source imaging can be used to localize and resolve a large number of focal, multi-focal, dipolar, and distributed neuronal sources and a variety of temporal profiles with uncorrelated, partially-correlated, as well as 100% correlated source time-courses. The disclosed VESTAL technology can be used to create regional-based normative databases for MEG slow-wave and MEG functional connectivity, e.g., in which these normative databases can be used to objectively detect brain injuries and abnormal neuronal networks in patients with neurological and/or psychiatric disorders, and can also include a built-in feature for across-subject registration and regional-based group analysis.

### BRIEF DESCRIPTION OF THE DRAWINGS

FIGS. 1A and 1B show block diagrams of exemplary frequency-based VESTAL processes.

FIG. 1C shows a diagram of an exemplary VESTAL system.

FIGS. 2A-2E show images of an exemplary processing stream of frequency-domain VESTAL MEG source imaging for slow-wave signals.

FIG. 3 shows a diagram of an exemplary empirical Kaplan-Meier cumulative distribution function of the Zₘₐₓ for MEG slow-wave signals.

FIG. 4 shows a data plot of exemplary Zₘₐₓ values obtained from frequency-domain VESTAL low-frequency source imaging.

FIG. 5 shows diagrams of exemplary cortical gray-matter areas that generate abnormal MEG slow-waves in individual patients from mild blast, mild non-blast, and moderate TBI groups.

FIG. 6A shows an exemplary diagram of abnormal slow-wave signal generation.

FIGS. 6B and 6C show exemplary data plots demonstrating comparative blast data in TBI groups.

FIG. 7 shows an exemplary covariance-matrix-based VESTAL MEG source imaging diagram.

Like reference symbols and designations in the various drawings indicate like elements.

### DETAILED DESCRIPTION

Magnetoencephalography (MEG) is a technique for mapping brain activity by recording magnetic fields produced by intracellular electrical currents in the brain. For example, synchronized electrical currents generated by neuron cells can produce magnetic fields. For example, these magnetic signals can originate from a net effect of ionic currents flowing between neurons (e.g., during synaptic transmission), which can be modeled as electric dipoles, e.g., currents having a position, orientation, and magnitude and other non-dipole current sources that produce magnetic signals. The magnetic fields produced by neurons exhibit magnitudes on the order of femto Teslas (fT), e.g., such as 10¹ fT for cortical activity and 10³ fT for a human alpha rhythm. These neuronal magnetic signals can be detected by sensitive magnetometers. However, these neuronal magnetic signals are relatively weak in comparison to typical ambient magnetic noise of the outer body environment (e.g., which can be on the order of nT to μT).

MEG techniques can be implemented to determine the location of neuronal signaling (e.g., electric activity of neurons) within the brain by detecting and analyzing magnetic field signals emitted by ionic currents in the brain using magnetic sensors surrounding the outside of a subject's skull and subsequently performing signal processing and analysis. Determining the location of neuronal signaling can be characterized as an inverse problem, in which model parameters (e.g., the location of the activity or source location) have to be estimated from the measured MEG data based on locations and spatial distribution of a given set of magnetic sensors. For example, one of the challenges of inverse problems is that an inverse problem may not have a unique solution. Therefore, to achieve a meaningful and accurate solution, possible solutions can be derived using models involving prior knowledge of brain activity. For example, MEG source modeling for analyzing MEG data can use equivalent current dipole models to fit operator-specified time-window of activities.

The disclosed technology includes techniques, devices, and systems for solving inverse problems including signal source imaging by employing a frequency-domain vector-based spatio-temporal analysis using L1-minumum norm solution (VESTAL). Implementations of the disclosed VESTAL technology can be used to determine source data and produce source images with high spatial and temporal resolutions from detected signals, e.g., in which the source data locations are substantially greater than the number of sensors used to detect the signals.

In one aspect, the disclosed frequency-domain VESTAL technology can be used for high resolution MEG source imaging that can be implemented in non-invasive diagnostic applications to detect and characterize loci of neuronal injury and abnormal neuronal networks, e.g., in patients with neurological and/or psychiatric disorders. For example, exemplary VESTAL techniques can be used to detect neuronal injuries and abnormal neuronal networks by employing neuroimaging and brain activity mapping using a high resolution MEG method.

The disclosed frequency-domain VESTAL technology can also be implemented in high resolution source imaging techniques to recover source information from other types of sensor arrays and signal data, e.g., the sensors including, but not limited to, radar, sonar, astronomical telescopes, magnetotelluric sensors, oceanographic sensors, optical sensor arrays, and other electromagnetic sensor arrays, among others.

Exemplary implementations of the disclosed VESTAL technology in MEG source imaging applications that utilized slow-wave MEG measurements (e.g., in a frequency range of 1-4 Hz) to identify neurological disorders are described. For example, a frequency-domain VESTAL source imaging technique using oscillatory MEG signals was implemented in patients with mild TBI (mTBI), medium TBI, and no TBI. TBI is a leading cause of sustained physical, cognitive, emotional, and behavioral deficits among members of a civilian population and military personnel (e.g., which can be due to motor vehicle accidents, sports-related concussions, falls, assaults, and blast-related traumas, among other incidents). Although post-concussive symptoms (PCS) in mTBI can be resolved by three months after the trauma in the majority of mTBI cases, a substantial amount of mTBI subjects (e.g. about 20%, varying from 8% to 33%) exhibit persistent long-term cognitive and/or behavioral impairments. Additionally, there have been few effective treatments for mTBI, and conventional neuroimaging techniques (e.g., such as CT, MRI, and DTI) have limited sensitivity to detect physiological alterations caused by TBI. MEG-based imaging techniques can provide MEG data of neuronal activity across a frequency spectrum. For example, frequencies above 8 Hz can be associated with normal neurological activity, but injured neuronal tissues (e.g., due to head trauma, brain tumors, stroke, etc.) may generate abnormal focal or multi-focal low-frequency neuronal magnetic signal in the delta band (e.g., 1-4 Hz) or the theta band (e.g., 5-7 Hz), which can be directly measured and localized using the disclosed MEG-based VESTAL techniques.

In one example, an imaging (lead-field) data set can be taken, in which the source space (e.g., gray-matter brain volume) is divided into a grid of source locations. Exemplary MEG time-domain signals can then be expressed in a data matrix, e.g., such as B(t)=[b(t₁),b(t₂), . . . ,b(t_{N})], where N is the number of time samples and b(tᵢ) is a M×1 vector containing the magnetic fields at M sensor sites at time point tᵢ. The data matrix can be expressed as:

B(t)=GQ(t)+Noise(t) (Eq. 1)

where G can represent an M×2P gain (lead-field) matrix calculated from MEG forward modeling for the pre-defined source grid with P dipole locations, e.g., with each dipole location having two orthogonal orientations (e.g., θ and φ), and Q(t) can represent a 2P×N source time-course matrix. In the exemplary spherical MEG forward head model, θ and φ can represent the two tangential orientations for each dipole location; whereas in a realistic MEG forward model using the boundary element method (BEM), the θ and φ orientations can be obtained as the two dominant orientations from the singular value decomposition (SVD) of the M×3 lead-field matrix for each dipole. An exemplary inverse solution in Eq. 1 can be to obtain the source time-courses Q(t) for given MEG sensor wave-forms B(t). For example, for each time-sample, since the number of unknown parameters can be far greater than the number of sensor measurements (e.g., 2P>>M), MEG source imaging deals with a highly under-determined problem, e.g., in which there can be a large number of solutions that will fit the data. To reduce the ambiguity, additional constraints (e.g., source models) can be applied, as described herein.

The disclosed vector-based spatio-temporal analysis using L1-minimum norm (VESTAL) techniques can be implemented as a high-resolution time-domain and frequency-domain MEG source imaging solution for Eq. 1 that includes the following exemplary properties. For example, exemplary VESTAL techniques can be used to model many dipolar and non-dipolar sources; the disclosed VESTAL techniques can be implemented with no pre-determination of the number of sources (e.g., model order); and exemplary VESTAL techniques can resolve 100% temporally correlated sources. For example, to more effectively image oscillatory MEG signals, such as complicated MEG slow-waves, the described VESTAL techniques can be utilized in the frequency-domain. For example, the MEG signal for a few frequency bins can be analyzed, instead of thousands of time samples in a given time window (e.g., an epoch).

For example, to image resting-state MEG signal, the spontaneous time-domain data (e.g., MEG signal data) can be divided into epochs. For example, by performing Fast Fourier Transform (FFT) techniques to transfer each epoch into F frequency bins, Eq. 1 can be expressed as:

└Kᵣₑₐₗ(f)K_{imag}(f)┘=G└Ωᵣₑₐₗ(f)┘ (Eq. 2)

where the M×F matrices Kᵣₑₐₗ and K_{imag} are the real and imaginary parts of the FFT of the sensor waveform B(t) for given frequency f, and the 2P×F matrices Ωᵣₑₐₗ and Ω_{imag} contain the Fourier Transformation coefficients of source time-course Q(t). For example, the inverse solution to the frequency-domain Eq. 2 can include determining Ωᵣₑₐₗ and Ω_{imag}, which are the source amplitudes at different frequency bins for given sensor-space frequency-domain signal Kᵣₑₐₗ and K_{imag}. As in the time-domain, the exemplary inverse problem can be under-determined.

For example, letting ω be the 2P×1 source-spaced Fourier coefficient vector from a column in either Ωᵣₑₐₗ or Ω_{imag} for a given frequency bin (e.g., no longer represented with the “real” and “imag” subscripts for now), and letting G=USV^{T} be the truncated singular value decomposition of the gain matrix, the L1-minimum norm solution to Eq. 2 can be represented as:

min(w^{T}|ω|) subject to constraints SV^{T}ω≅U^{T}κ (Eq. 3)

where κ is the sensor-spaced Fourier coefficient vector from the corresponding column in either Kᵣₑₐₗ or K_{imag}. For example, on an exemplary Elekta/Neuromag VectorView system, the top 40 singular values can be kept during the singular value decomposition (SVD) truncation of the gain matrix G. In Eq. (3), w is a 2P×1 weighting vector chosen to remove potential bias towards grid nodes at the superficial layer, and it can be taken to be the column norm of the G matrix or a Gaussian function. The solution to Eq. (2) can be a non-linear minimization procedure since the source-space Fourier coefficient ω can be either positive or negative. However, in practice, one can replace the absolute values in |ω| with the following two sets of non-negative values related to ω, and solve the set of equations through linear programming (LP). For example, with the introduction of two new non-negative variables ω^{a} and ω^{b}, Eq. (3) can be represented as:

min(w^{T}(ω^{a}+ω^{b})) subject to SV^{T}ω≅U^{T}κ,ω=ω^{a}−ω^{b},

{ωⱼ^{a}},{ωⱼ^{b}}≧0,{ωⱼ},j=1,2, . . . ,2P (Eq. 4)

Eq. (4) can be solved (e.g., by using LP techniques, including SeDuMi to solve the above equation-set to get source imaging ω for a given frequency bin). This exemplary step can be repeated for each frequency bin to obtain the whole frequency-domain source images for both the real and imaginary parts of the signal, e.g., ωᵣₑₐₗ or ω_{imag}.

The L1-minimum norm approach can be used to address a problem in which the solution can have a small tendency (bias) towards the coordinate axes. For example, in a spherical MEG head model, for a dipole at the i^{th} node of the grid, the vector-based L1-minimum norm solution can also be expressed as minimizing

$${\sum }_{i = 1}^{P} {w}_{i} {ω}_{i} ( \vert cos ( {ψ}_{i} ) \vert + \vert sin ( {ψ}_{i} ) \vert ) ,$$

where ψᵢ is the angle between total dipole moment and the orientation of the elevation in a tangential plane containing the dipole node, and ωᵢ=\sqrt{(ωᵢ^{θ})²+(ωᵢ^{φ})²} is the non-negative dipole strength. This can introduce a bias towards the coordinate axes. In order to handle this small bias, an additional correction factor (|cos(ψᵢ^{e})|+|sin(ψᵢ^{e})|)⁻¹ can be included in the weighting vector w in Eq. (4) for one more iteration, where ψᵢ^{e} is the angle associated with the estimated orientation based on L1-minimum norm solution without the correction factor.

In a conventional time-domain L1-norm approach, problems can exist that include instability in spatial construction and discontinuity in reconstructed source time-courses. For example, this can be seen as “jumps” from one grid point to (usually) the neighboring grid points. Equivalently, the time-course of one specific grid point can show substantial “spiky-looking” discontinuity. Direct frequency-domain L1-norm solution (e.g., Ωᵣₑₐₗ or Ω_{imag}) operating on individual frequency bins can also suffer from the same instability as in the time domain.

For example, according to MEG physics, magnetic waveforms in the sensor-space are linear functions of the dipole time-courses in the source-space. The exemplary frequency-domain VESTAL can include performing singular value decomposition (SVD) for the M×F frequency domain MEG sensor signal:

K=U_{B}S_{B}V_{B}^{T} (Eq. 5)

(e.g., variables in Eq. 5 are shown without the “real” and “imag” subscripts, as it applies to both).

For example, all frequency-related information in the MEG sensor signal can be represented as a linear combination of the singular vectors in the matrix V_{B}. For example, since MEG sensor-spaced signals can be linear functions of the underlying neuronal source-space signal, the same signal sub-space that expands the frequency dimension of sensor-space Fourier coefficient matrix K can also expand the frequency dimension of the 2P×F source-space Fourier coefficient matrix Ω (e.g., also noted that the “real” and “imag” subscripts are not shown here). For example, by projecting Ω towards V_{B} it is ensured that the source spectral matrix Ω and sensor spectral matrix K share the same frequency information (e.g., as required by the MEG physics):

Ω_{Freq}_{_{—}}_{VESTAL}=ΩP_{∥} (Eq. 6)

where the projection matrix P_{∥}=V_{B} V_{B}^{T} is constructed using the dominant (signal-related) temporal singular vectors (subspace) of the sensor waveforms. For example, Ω_{Freq}_{_{—}}_{VESTAL} called the frequency-domain singular vectors (subspace) of the sensor waveforms. Ω_{Freq}_{_{—}}_{VESTAL} can be referred to as the frequency-domain VESTAL solution. For example, the procedure as described in Eqs. (4)-(6) can apply to the real and imaginary parts of the signal separately. The exemplary frequency-domain VESTAL source image can be obtained by combining the real and imaginary parts together.

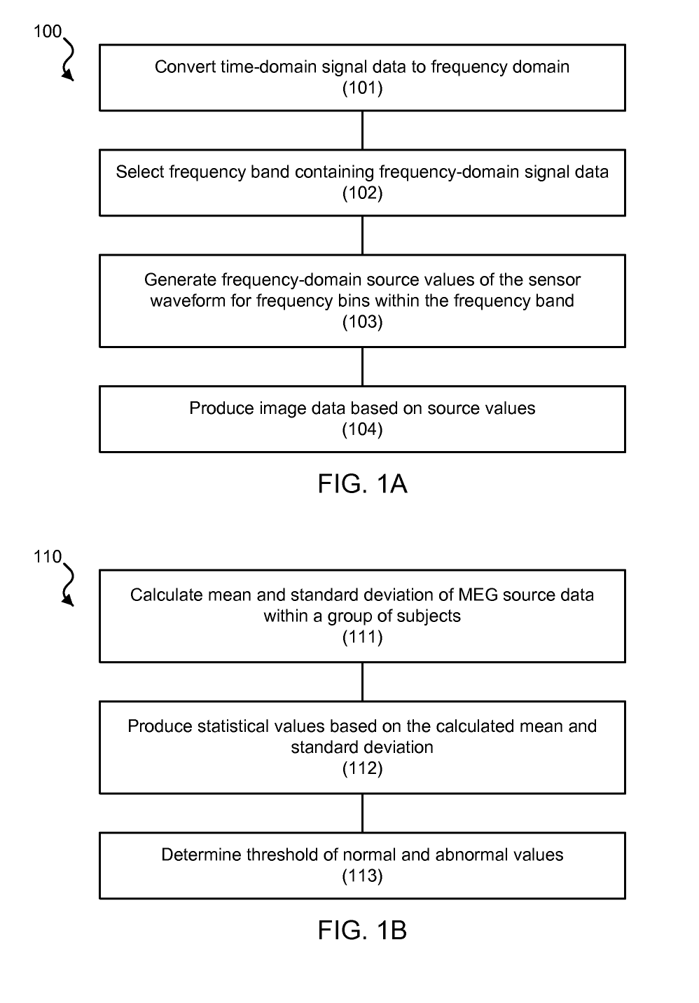

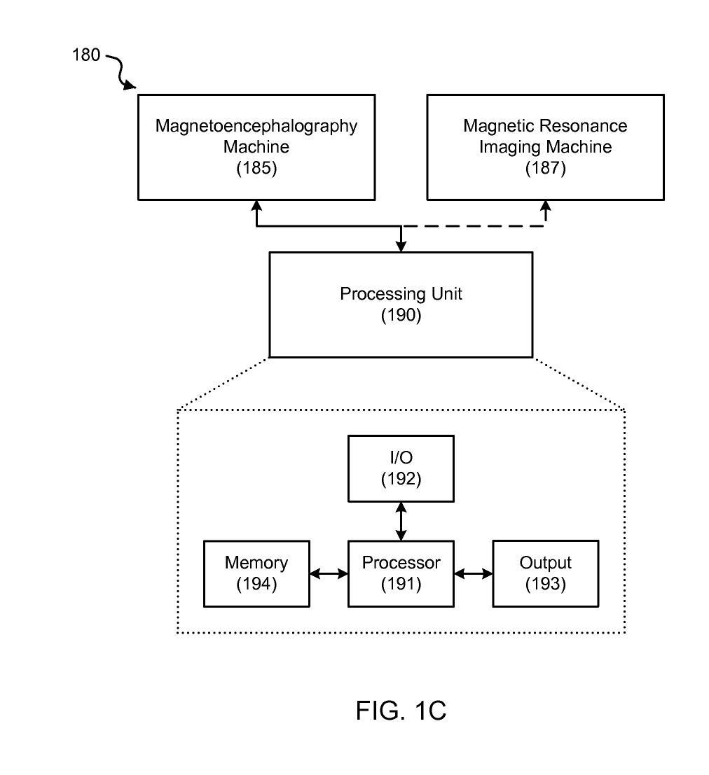

FIGS. 1A and 1B show block diagrams of exemplary frequency-based VESTAL processes. FIG. 1A shows an exemplary process 100 to determine source data with high spatial and temporal resolutions from detected signal data, e.g., in which the source data locations are substantially greater (e.g., at least 10 times greater) than the number of sensors used to detect the signals. For example, the process 100 can be used to implement a frequency-domain VESTAL technique for MEG source imaging that can select signal data (e.g., magnetic field signals obtained by MEG sensors) within one or more frequency bands from a spectrum of the signal data in the frequency domain, define location values (e.g., source grid points that can correspond to voxels) that map to locations within the brain, and generate a source value of signal power based on the selected signal data corresponding to the location values for each frequency bin of the selected frequency band. For example, selecting the signal data within the particular frequency band can include removing other signal data associated with other frequency bands, e.g., optimizing the generation of signal source values.

The exemplary process 100 can include a process 101 to convert time-domain signal data to data in the frequency domain. For example, the process 101 can include implementing a Fourier Transformation to convert the time-domain MEG sensor waveforms to the frequency domain and obtain the Fourier components (e.g., the exemplary sensor-space frequency-domain signal Kᵣₑₐₗ and K_{imag}) of the MEG sensor waveforms, as described by Eq. 2.

The exemplary process 100 can include a process 102 to select a specific frequency band or multiple frequency bands. For example, the process 102 can include selecting frequency-domain MEG signal data in the delta band (e.g., 1-4 Hz). For example, the selected frequency band(s) can include any number of discrete frequencies (e.g., which can be referred to as frequency bins), e.g., such as 1.0, 1.1, 1.2, . . . 4.0 Hz within the exemplary selected delta band. For example, frequency-domain signal data can be selected by determining the particular frequency bands, e.g., by filtering the signal data through one or more filters (e.g., including low pass, high pass, band pass filters, among other filters).

The exemplary process 100 can include a process 103 to generate frequency-domain singular vectors of the sensor waveform (e.g., the frequency-domain VESTAL solutions), e.g., by applying minimum L1-norm inverse solution. For example, the exemplary singular vectors of the sensor waveform can include the source value of signal power based on the selected MEG signal data corresponding to each source location (e.g., voxels in an image) for each frequency bin within the selected frequency band. For example the process 103 can include calculating the MEG forward solution using a boundary element method (BEM) to construct the gain matrix G (e.g., also referred to as the lead-field matrix), and applying singular value decomposition (SVD) to the gain matrix G=USV^{T}. The process 103 can include arranging the exemplary SVD matrices of G and the Fourier components of sensor waveforms (Kᵣₑₐₗ and K_{imag}), as described in Eqs. 3 and 4, for minimum L1-norm solver. For example, the first terms of the L1-minimum norm requirement (e.g., min(w^{T}|ω|) in Eqs. 3 and 4) are the important terms to obtaining high-resolution source imaging, e.g., for MEG source imaging that includes the number of MEG sensors (e.g., ˜300) that is far less than the number of source variables (e.g., ˜>10,000) for a typical sources grid with thousands of voxels (e.g., ˜10,000 voxels). For example, the remaining terms of Eqs. 3 and 4 are to ensure that the solutions fit the MEG data, e.g., in terms of Fourier components of sensor waveforms. The process 103 can include using linear-programming techniques as the minimum L1-norm solver, e.g., to solve Eq. 4 and obtain the source-space Fourier coefficient ω, e.g., the current flow vectors for each voxel of MEG source current images.

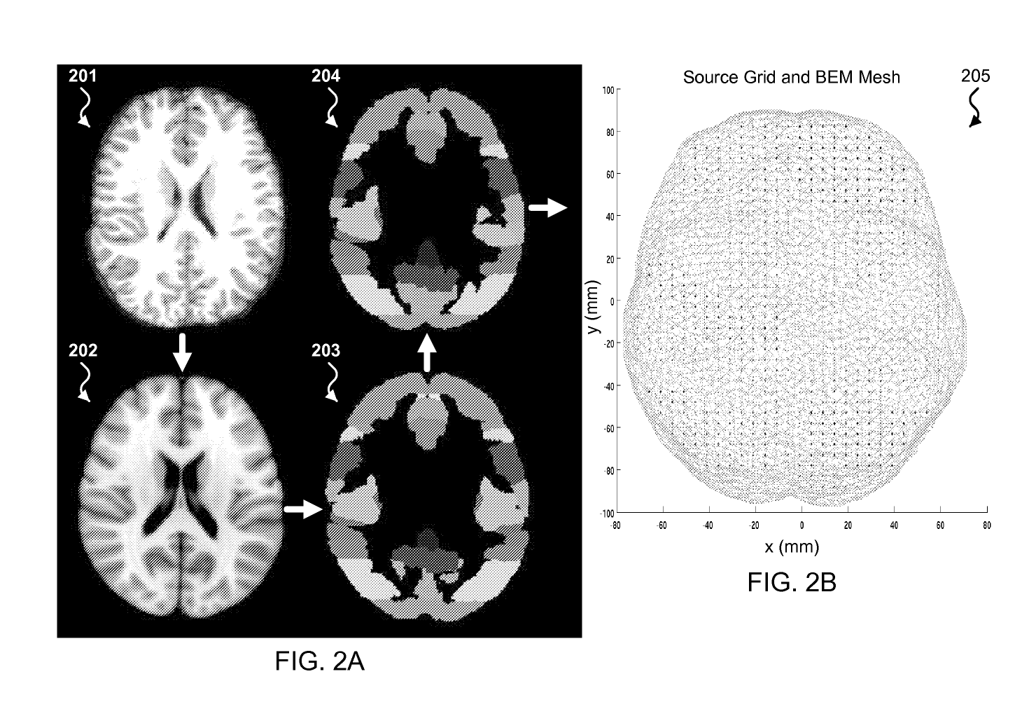

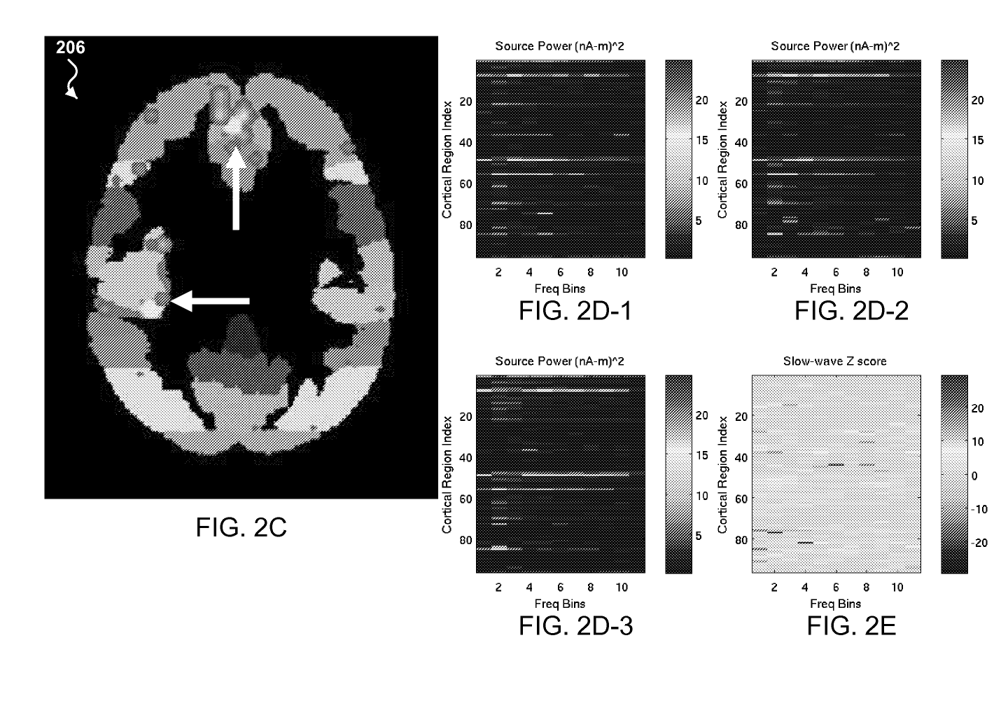

The exemplary process 100 can include a process 104 to produce image data based on the source values (e.g., the source-space coefficients). For example, the frequency-domain VESTAL solutions can be used to produce source images representing MEG source power (e.g., within each voxel of an image, which can include ˜10,000 voxels). The exemplary MEG source imaging diagram can be an MEG spatial map of the source values having a high resolution, e.g., a resolution of at least one source value per one millimeter volume of the brain. In some examples, the resolution of the MEG spatial map can be 2 mm to 3 mm, e.g., which can based on the signal-to-noise ratio. For example, the process 104 can include removing form systematic bias and constructing the VESTAL source power images for each frequency bins (e.g., in accordance with Eqs. 5 and 6). For example, the process 104 can include displaying the VESTAL-based MEG source power images on exemplary anatomical MRI images (e.g., of the brain). For example, an exemplary mask (e.g., such as brain cortical region mask) can be applied to group the exemplary MEG source power data from each source location (e.g., the exemplary ˜10,000 voxels) into a smaller number of regions (e.g., such as 96 cortical regions, as shown later in FIG. 2A) to develop exemplary 2D MEG frequency-power diagrams (e.g., including matrix dimensions: number of brain regions×number of frequency bins)

The disclosed VESTAL technology can be implemented in non-invasive diagnostic applications to detect and characterize loci of neuronal injury and abnormal neuronal networks, e.g., in patients with neurological and/or psychiatric disorders. FIG. 1B shows an exemplary process 110 to create a normative database that can be used to characterize and distinguish healthy and abnormal brains. For example, the exemplary process 100 can be implemented for MEG source imaging in a large number of healthy subjects (e.g., subjects without brain injury, disease, or disorder) to develop a healthy control data base for each cell of the exemplary 2D MEG frequency-power diagrams. As shown in FIG. 1B, the process 110 can include a process 111 to calculate the mean and standard deviation for each cell of an exemplary 2D MEG frequency-power diagrams across subjects within a group, e.g., such as the healthy control subjects. The process 110 can include a process 112 to produce statistical score values (e.g., referred to as Z-score values) based on the calculated mean and standard deviation values of the exemplary group. For example, the process 112 can include converting the 2D MEG frequency-power diagram of each healthy control subject into a Z-score 2D diagram based on the group mean and standard deviation for each cell. The process 110 can include a process 113 to determine a threshold value that can be used to differentiate between normal and abnormal values. For example, the process 113 can include selecting the highest Z-value for the entire Z-score diagram of each control, and designating that Z-value to represent that control's maximum Z-score. For example, the highest maximum Z-score of all of the controls can be chosen, e.g., by setting that value as the threshold to differentiate between normal (e.g., less than or equal to that threshold Z-score) vs. abnormally-high delta power (e.g., higher than that threshold Z-score). For example, the exemplary process 100 can be implemented for MEG source imaging in a large number of subjects with neurological or psychiatric disorders. Exemplary 2D MEG frequency-power diagrams of these exemplary subjects can be converted into Z-score 2D diagram based on the determined threshold, and regions with Z-scores exceeding the threshold (e.g., established in the healthy control database) can be identified.

FIG. 1C shows an exemplary MEG-based system 180 for implementing the disclosed VESTAL techniques, e.g., determining non-invasive, in vivo biomarker data of healthy and diseased tissue using the high resolution MEG source imaging technique in time and frequency domains to detect loci of neuronal injury and abnormal neuronal networks. FIG. 1C shows one aspect of the exemplary system 180 that can include a magnetoencephalography machine 185 magnetic resonance imaging machine 187, which can be controlled by a processing unit 190. For example, the exemplary processing unit 190 can be used to implement the process 100 and other processes of the disclosed VESTAL technology.

The exemplary MEG machine 185 can be used in the system 180 to implement magnetic field signal data acquisition. For example, the exemplary MEG machine 185 can include an array of magnetometer sensors that can detect magnetic signals emitted by the brain, e.g., a superconducting quantum interference device (SQUID) can be such a sensor. In some implementations, the SQUID sensors can be contained in a casing that can maintain cryogenic temperatures for operation, e.g., such as a helmet-shaped liquid helium containing vessel or dewar. The MEG machine 185 can include an array of hundreds or thousands of SQUIDS that can record simultaneous measurements over the head at several regions on a micrometer or millimeter scale. A large number of sensors can be used at different spatial locations around the brain to collect magnetic signals emitted by the brain to gain the spatial diversity of the brain emission of magnetic signals. As the number of the sensor increases, a better spatial resolution of the source imaging information can be achieved. The number of sources in the brain in implementing the present technology is greater than the number of sensors. The present technology allows use of the limited number of sensors to provide MEG imaging at a much greater number of source locations in the brain. The system 180 can include a magnetically shielded room to contain the exemplary MEG machine 185 to minimize interference from external magnetic noise sources, e.g., including the Earth's magnetic field, electrical equipment, radio frequency (RF) signaling, and other low frequency magnetic field noise sources. The exemplary magnetically shielded room can be configured to include a plurality nested magnetically shielding layers, e.g., including pure aluminum layer and a high permeability ferromagnetic layer (e.g., such as molybdenum permalloy).

The exemplary MRI machine 187 can be used in the system 180 to implement an MRI imaging in support of the exemplary VESTAL characterization process under the control of the exemplary processing unit 190. The MRI machine 187 can include various types of MRI systems, which can perform at least one of a multitude of MRI scans that can include, but are not limited to, T1-weighted MRI scans, T2-weighted MRI scans, T2*-weighted MRI scans, spin (proton (¹H)) density weighted MRI scans, diffusion tensor imaging (DTI) and diffusion weighted imaging (DWI) MRI scans, diffusion spectrum imaging (DSI) MRI scans, Tip MRI scans, magnetization transfer (MT) MRI scans, real-time MRI, functional MRI (fMRI) and related techniques such as arterial spin labeling (ASL), among other MRI techniques.

The exemplary processing unit 190 can include a processor 191 that can be in communication with an input/output (I/O) unit 192, an output unit 193, and a memory unit 194. For example, the processing unit 190 can be implemented as one of various data processing systems, such as a personal computer (PC), laptop, tablet, and mobile communication device. To support various functions of the processing unit 190, the exemplary processor 191 can be included to interface with and control operations of other components of the processing unit 190, such as the exemplary I/O unit 192, the exemplary output unit 193, and the exemplary memory unit 194.

To support various functions of the processing unit 190, the memory unit 194 can store other information and data, such as instructions, software, values, images, and other data processed or referenced by the processor 191. For example, various types of Random Access Memory (RAM) devices, Read Only Memory (ROM) devices, Flash Memory devices, and other suitable storage media can be used to implement storage functions of the memory unit 194. The exemplary memory unit 194 can store MEG and MRI data and information, which can include subject MEG and MRI data including temporal, spatial and spectral data, MEG system and MRI machine system parameters, data processing parameters, and processed parameters and data that can be used in the implementation of a VESTAL characterization. The memory unit 194 can store data and information that can be used to implement an MEG-based VESTAL process and that can be generated from an MEG-based VESTAL characterization algorithm and model.

To support various functions of the processing unit 190, the exemplary I/O unit 192 can be connected to an external interface, source of data storage, or display device. For example, various types of wired or wireless interfaces compatible with typical data communication standards, such as Universal Serial Bus (USB), IEEE 1394 (FireWire), Bluetooth, IEEE 802.111, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), and parallel interfaces, can be used to implement the I/O unit 192. The I/O unit 192 can interface with an external interface, source of data storage, or display device to retrieve and transfer data and information that can be processed by the processor 191, stored in the memory unit 194, or exhibited on the output unit 193.

To support various functions of the processing unit 190, the output unit 193 can be used to exhibit data implemented by the exemplary processing unit 190. The output unit 193 can include various types of display, speaker, or printing interfaces to implement the exemplary output unit 193. For example, the output unit 193 can include cathode ray tube (CRT), light emitting diode (LED), or liquid crystal display (LCD) monitor or screen as a visual display to implement the output unit 193. In other examples, the output unit 193 can include toner, liquid inkjet, solid ink, dye sublimation, inkless (such as thermal or UV) printing apparatuses to implement the output unit 193; the output unit 193 can include various types of audio signal transducer apparatuses to implement the output unit 193. The output unit 193 can exhibit data and information, such as patient diagnostic data, MEG machine system information, MRI machine system information, partially processed MEG-based VESTAL processing information, and completely processed MEG-based VESTAL processing information. The output unit 193 can store data and information used to implement an exemplary MEG-based VESTAL characterization process and from an implemented MEG-based VESTAL characterization process.

Exemplary implementations were performed using the disclosed VESTAL technology in an automated and operator-independent MEG low-frequency source imaging technique that can be applied for diagnosing mild TBI.

For example, the disclosed VESTAL technology was used to characterize the relationship between the generation of abnormal MEG delta-waves and potential reduction of MEG functional connectivity in beta and gamma bands in mild TBI patients. For example, gray-matter neurons that experience deafferentation due to axonal injury can cause the generation of abnormal delta-wave at low frequency and the reduction of cortico-cortical coherence at higher frequency (beta and gamma bands). Twenty-five mild TBI patients and twenty-one healthy control subjects participated in these exemplary implementations, and resting-state MEG signals with eyes-open and eyes-closed were recorded. Also investigated in the exemplary implementations was the neurophysiological basis of TBI-related cognitive impairments using an N-back working memory MEG task in mild TBI patients. For example, to examine the resting-state functional connectivity in highly correlated neuronal networks using MEG, a regional-based connectivity analysis using the Dual-core Beamformer was developed. The results of the exemplary implementations demonstrated: (1) that in resting-state MEG examination of the mild TBI patients, the brain areas that generated abnormal MEG delta-waves also show reduced functional connectivity with other brain regions in the beta and gamma bands; (2) the reduced functional connectivity in the working memory network in resting-state examination correlated with the results of the N-back working memory exam in mild TBI patients; and (3) the exemplary MEG findings are consistent with post-concussive symptoms and results of neuropsychological exams in mild TBI.

Exemplary data acquisition and signal pre-processing protocols employed in the exemplary implementations of MEG source imaging using the disclosed VESTAL technology are described. For example, resting-state MEG data (e.g., spontaneous recording for detecting low-frequency MEG signals) were collected using an Elekta-Neuromag VectorView™ whole-head MEG system with 306 MEG channels in a multi-layer magnetically-shielded room (IMEDCO-AG). The exemplary recording was divided into three 5-minute blocks with eyes closed, e.g., alternating with three 5-minute blocks with eyes open and the subjects watching a fixation point. The order of blocks was counter-balanced between subjects. Exemplary data were sampled at 1000 Hz and were run through a high-pass filter with 0.1 Hz cut-off and a low-pass filter with 330 Hz cut-off. Eye blinks, eye movements, and heart signals were monitored.

The exemplary MEG data are first run through MaxFilter to remove external interferences (e.g., magnetic artifacts due to metal objects, strong cardiac signals, environment noises, etc.), and correct for head movement. Next, residual artifacts near the sensor array due to eye movements and residual cardiac signals were removed using Independent Component Analysis (e.g., customized software of ICALAB). For example, the EKG artifacts in the MEG data were also removed when the MEG data were passed through MaxFilter.

Exemplary MEG-MRI registration and BEM forward calculations employed in the exemplary implementations of MEG source imaging using the disclosed VESTAL technology are described. Structural T1-weighted 3D MR images of the head of exemplary subjects can be collected using any MRI scanner. For example, structural MR images of the exemplary subjects' heads were collected using a General Electric 1.5T Excite MRI scanner (ver. 14 software release). The acquisition contained a standard high-resolution anatomical volume with a resolution of 0.94×0.94×1.2 mm³ using a T1-weighted 3D-IR-FSPGR pulse sequence. For example, to co-register the MEG with MRI coordinate systems, three anatomical landmarks (e.g., left and right pre-auricular points, and nasion) were measured for each subject using the Probe Position Identification system (Polhemus, USA). By identifying the same three points on the subject's MR images, a transformation matrix involving both rotation and translation between the MEG and MR coordinate systems was generated. For example, to increase the reliability of the MEG-MR co-registration, approximately 80 points on the scalp were digitized with the Polhemus system, in addition to the three landmarks, and those points were co-registered onto the scalp surface of the MR images. The MEG-MR co-registration error was expected to be less than 3 mm. For example, the T1-weighted images were also used to extract the innermost skull surface (SEGLAB from Elekta/Neuromag). The innermost skull surface was used to construct a realistic head model for MEG forward calculation based on a boundary element method (BEM) technique. Also for example, in addition to the exemplary T1-weighted MRI, the following MRI sequences were performed, e.g., such as axial T2*-weighted; axial fast spin-echo T2-weighted; axial FLAIR; and axial DWI.

An exemplary processing stream of frequency-domain VESTAL source imaging for MEG slow-wave signals is described. For example, each of the artifact-free, 5-minute long, eyes-closed, resting-state MEG sensor-space data were run through a band-pass filter with the passing band at 1-4 Hz (e.g., delta-frequency band) and transition bands (e.g., Hanning Windows) of 0.5-1 Hz and 4-6 Hz, respectively. Then, for example, the exemplary sensor-space MEG data were divided into 2.5-second epochs with 50% overlap in time. For each exemplary epoch, an FFT was performed to obtain the sensor-space FFT coefficients Kᵣₑₐₗ and K_{imag} for 11 equally-spaced low-frequency bins with center-frequencies between 0.98 Hz and 5.86 Hz. These exemplary sensor-space frequency-domain data were used by the frequency-domain VESTAL to obtain the MEG low-frequency source images.

The source grid used in the exemplary implementation of the exemplary frequency-domain VESTAL technique was obtained by sampling the gray-matter areas from the exemplary T1-weighted MRI data of each subject. FIG. 2A includes T1-weighted MR images 201, 202, 203, and 204, e.g., which were registered to a standard atlas (e.g., MNI-152) using registration programs in a comprehensive library of analysis tools (e.g., such as FSL). As shown in FIG. 2A, the image 201 shows an exemplary individual subject's T1-weighted MRI data; the image 202 shows an exemplary MNI-152 Brain Atlas; the image 203 shows an exemplary Harvard-Oxford cortical region mask in the MNI-152 coordinate; and the image 204 shows the cortical region mask transferred to the individual subject's MRI coordinate. For example, the cortical, subcortical, and cerebellum gray-matter masks with pre-defined brain regions from the standard atlas can be transferred to the individual subject's coordinates (e.g., as shown in the image 204), e.g., using the inverse of the transformation in the first step. For example, the Harvard-Oxford Atlas, as part of the FSL software with masks of 96 cortical gray-matter regions (e.g., 48 in each hemisphere), 15 sub-cortical gray-matter regions, and cerebellum, can be used in this exemplary process.

For example, the regional masks in this exemplary subject were re-sampled to a cubic source grid with 5 mm size for frequency-domain VESTAL analysis. FIG. 2B shows an image 205 demonstrating the exemplary cortical regions that are re-sampled to the exemplary MEG source grid. For example, a realistic BEM head model was used for MEG forward calculation, e.g., with the BEM mesh (e.g., shown as gray triangles in the image 205) obtained from tessellating the inner skull surface from the MRI into ˜6000 triangular elements with ˜5 mm size. These exemplary grid-point based frequency-domain VESTAL low-frequency source images demonstrate high spatial resolution and can be used to diagnose neurological disorders and pathologies. Yet, for example, for each low-frequency bin, a region-based MEG slow-wave diagram can be created by applying the cortical mask to the grid-point based frequency-domain VESTAL result.

An exemplary frequency-domain VESTAL analysis (e.g., as described in Eqs. (4)-(6)) was performed for the real and imaginary part of each epoch separately to obtain the frequency domain source imaging Ω_{Freq}_{_{—}}_{VESTAL}. For example, the selection of the signal-related subspace dimension of the V_{B} matrix equals the number of frequency bins within the passing-band of interest. For each grid point, the real and imaginary source images from the two perpendicular orientations (e.g., θ and φ)) were combined to create source-power images for each frequency bin for that epoch. This exemplary procedure was repeated for all epochs in the exemplary 5-minute eyes-closed resting-state data. For example, a set of 11 mean-source-power images (one for each low-frequency bin) were obtained, e.g., by averaging source-power images across all epochs. FIG. 2C shows an image 206 of the MEG slow-wave activities (e.g., image features on the image 206 referred to as “hot spots”), which was obtained by implementing the exemplary frequency-domain VESTAL technique. Exemplary yellow and red “hot spots”, highlighted by white arrows, show an example of the frequency-domain VESTAL slow-wave source-power image from one subject at one specific frequency bin. For example, the total power for one of the 96 cortical regions defined in the mask can be computed by summing up the slow-wave power from all grid points within each region.

The exemplary cortical mask (e.g., of the image 203) can then applied to these slow-wave activities. For example, FIGS. 2D-1, 2D-2, and 2D-3 show exemplary MEG slow-wave power diagrams (e.g., cortical regions versus frequency bins) for three healthy control subjects that were used to construct normative database (described later). FIG. 2E shows an exemplary Z-score diagram that demonstrates the comparative slow-wave power from a TBI patient with the normative database. In FIGS. 2D-1, 2D-2, and 2D-3, the exemplary data of increased slow-wave activities are shown in yellow and red color. For example, an important aspect for using MEG low-frequency source imaging to detect abnormalities in TBI patients can include the construction of a normative database. FIGS. 2D-1, 2D-2, and 2D-3 illustrate the exemplary procedure of developing a normative database based on the region-based slow-wave power diagrams containing 96 cortical gray-matter regions and 11 low-frequency bins. For example, in this exemplary procedure, 84 data sets from 28 healthy control subjects (e.g., three 5-minute eye-closed data sets per subject) were used. Two 96×11 (cortical region by frequency) power-frequency matrices for the low-frequency range were obtained in the normative database, e.g., one that contained the mean values by averaging across all 84 regional power-frequency diagrams, and another that contained the standard deviations. For example, although the exemplary source-grid used in this exemplary implementation of the frequency-domain VESTAL technique contained an additional 15 sub-cortical gray-matter areas and the cerebellum, the exemplary implementation focused on the 96 cortical gray-matter areas.

For example, any region-based power-frequency diagram in the low-frequency range from a testing subject can be converted into a Z-score diagram (96×11) for a mild TBI patient, demonstrated in FIG. 2E. FIG. 2E shows an exemplary Z-score diagram comparing the slow-wave power from a TBI patient with the normative database. In FIG. 2E, increased slow-wave activities are shown in yellow and red color. For example, each element of this Z-score diagram can be calculated by:

Zᵢⱼ=(Pᵢⱼ−Meanᵢⱼ^{ctrl})/SDᵢⱼ^{ctrl} i=1,2, . . . 96;j=1,2, . . . 11 (Eq. 7)

where Meanᵢⱼ^{ctrl} and SDᵢⱼ^{ctrl} are the mean and standard deviation values from the two 96×11 normative database matrices in healthy control subjects, containing the region-based power-frequency diagrams for the low-frequency range.

Exemplary maximum Z value statistical analyses and threshold setting (e.g., for assisting TBI diagnosis) were employed in the exemplary implementations of MEG source imaging using the disclosed VESTAL technology and are described. For example, an MEG slow-wave variable (measure) can be identified that shows minimum overlap between healthy controls and TBI patients. For example, in a TBI patient, at least one region can generate statistically abnormal slow-wave, regardless of the exact location of that region. The exemplary region-based MEG power-frequency diagram demonstrated the reduction of the family-wise error, e.g., due to multiple comparisons from thousands of grid points to 96 cortical gray-matter regions. Also, for example, data can be further processed to eliminate the likelihood of obtaining false-positive results. For example, the Z-value of slow-wave measurement can be used to further reduce the family-wise error and diagnose TBI.

The maximum Z-value of MEG slow-wave in a TBI patient can be used to differentiate individual TBI patients from the healthy control subjects. To demonstrate, for example, the Z-score diagrams (e.g., Eq. (7)) from the data sets in all healthy control subjects were calculated. For each Z-score diagram, the maximum Z-value across the exemplary 96 cortical regions and 11 frequency-bins was identified. The maximum Z-value (e.g., Zₘₐₓ) among three Z-score diagrams associated with three 5-minute eye-closed resting-state datasets was obtained for each healthy control and TBI subject. By plotting out the cumulative distribution function (CDF) of the exemplary maximum Z-values (Zₘₐₓ) from all healthy control subjects, a normative threshold can be obtained. This normative threshold can be used to identify individual TBI patients with abnormally high MEG slow-wave power on a statistical basis.

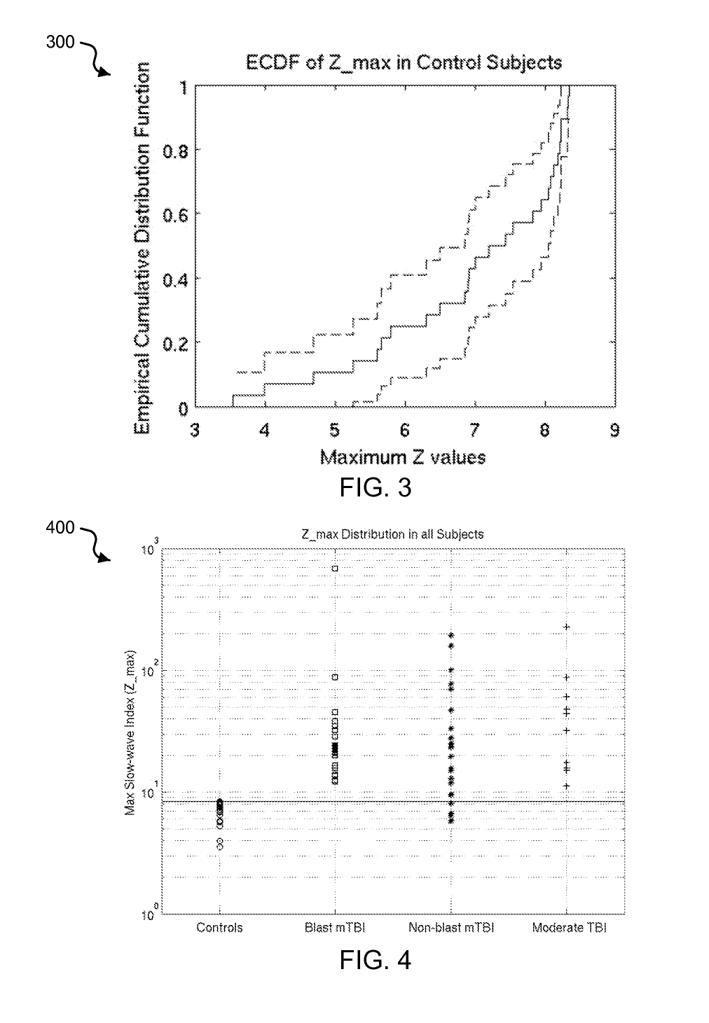

An exemplary normative database to determine the threshold for abnormal slow-wave power is described. For example, a threshold of the abnormal slow-wave source power was determined using the information from the normative database in healthy control subjects. FIG. 3 shows a diagram 300 demonstrating empirical (Kaplan-Meier) cumulative distribution function (ECDF) of the Zₘₐₓ for MEG slow-wave (e.g., the solid line in the diagram 300) from 28 healthy control subjects in the normative database. For example, in each subject, Zₘₐₓ represents the maximum value in the Z-score diagram obtained from the frequency-domain VESTAL across all of the exemplary 96 cortical gray-matter regions, 11 low-frequency bins, and three 5-min resting-state recordings with eyes-closed. The two exemplary dashed lines in the diagram 300 are the lower and upper bounds of the ECDF. All (100%) of the healthy control subjects exhibited their Zₘₐₓ values less than 8.36. For example, the exemplary Zₘₐₓ value of 8.36 was selected to be the threshold, e.g., as no healthy control subject showed Zₘₐₓ above this level.

FIG. 4 shows a data plot 400 of the Zₘₐₓ values, obtained from frequency-domain VESTAL low-frequency source imaging, plotted separately for 1) healthy control, 2) mild blast-related TBI, 3) mild non-blast-related TBI, and 4) moderate TBI groups. The y-axis of the data plot 400 is shown in logarithmic scale because some TBI patients showed markedly high slow-wave powers which translated into markedly high Zₘₐₓ values. One exemplary finding shown in the data plot 400 includes the low overlap of the Zₘₐₓ values between each TBI group and the healthy control group, e.g., with the patients in all TBI groups showing markedly higher slow-wave Zₘₐₓ values than the healthy control subjects. This property provides the basis of implementing the exemplary frequency-domain VESTAL technology in MEG low-frequency source imaging for the diagnosis of TBI.

For example, utilizing the 8.36 value of the exemplary Zₘₐₓ threshold, the correct positive finding rates were shown to be 96% for mild blast TBI patients (e.g., 24 out of 25), 82% for the mild non-blast TBI patients (e.g., 18 out of 22), and 100% for the moderate TBI patients (e.g., 10 out of 10). After the blast and non-blast mild TBI groups were combined together, the diagnostic rate was determined to be ˜90% for the combined mild TBI group.

The automated MEG low-frequency source imaging process produced highly significant differences between each TBI group and the healthy control group. For example, ANOVA analyses were performed, and the exemplary ANOVA results confirmed that in comparison to the healthy control group, the logarithm of Zₘₐₓ values are indeed significantly higher in the mild blast TBI (F=53.0, p<10⁻⁸, df=50), mild non-blast TBI (F=37.3, p<10⁻⁶, df=49), and moderate TBI (F=78.8, p<10⁻⁹, df=37) groups. In these exemplary ANOVA analyses, no significant differences in the logarithm of Zₘₐₓ values between the different TBI groups were shown.

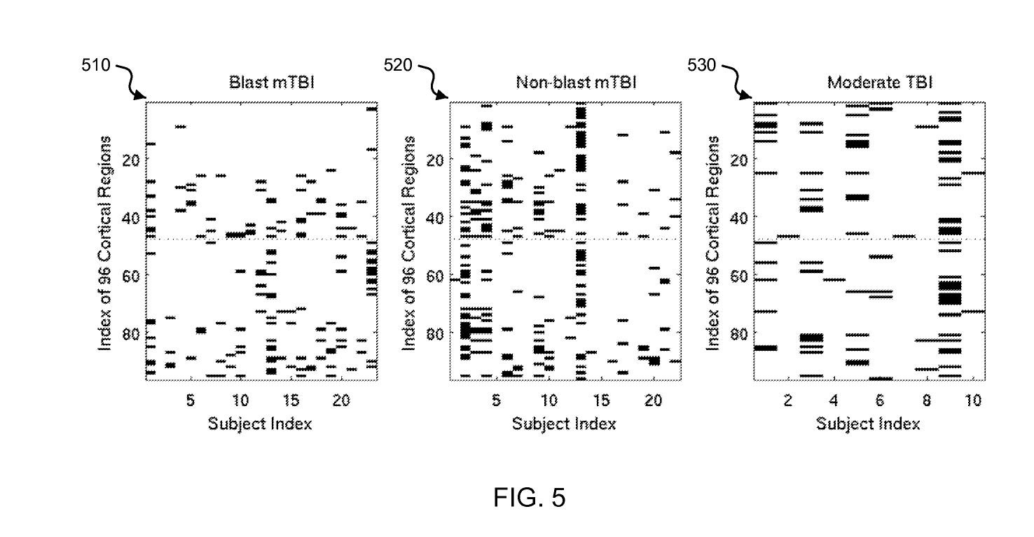

FIG. 5 shows diagrams 510, 520, and 530 showing cortical gray-matter areas (y-axis) that generate abnormal MEG slow-waves in individual patients (x-axis) from the mild blast TBI groups (diagram 510), mild non-blast TBI groups (diagram 520), and moderate TBI groups (diagram 530). For each subject (each column in the exemplary diagrams 510, 520, and 530), the black bars indicate the abnormal slow-wave generations that are beyond the threshold. In each diagram in FIG. 5, the regions in the left hemisphere (e.g., Regions 1-48) are separated from the analogous regions in the right hemisphere (e.g., Regions 49-96) by the dotted line. The majority of patients showed at least one, and often many cortical gray-matter areas that generated significant slow-waves. This exemplary data can demonstrate that the disclosed VESTAL technology can be used to characterize the loci and patterns of abnormal slow-wave generation in subjects with brain injury, disease, or disorder (e.g., as shown with the exemplary TBI patients).

For example, each of the exemplary diagrams 510, 520, and 530 in FIG. 5 can be analyzed in different ways, e.g., including across subjects and across different gray-matter regions. In the exemplary across-subject group analysis, the number of cortical gray-matter regions that showed abnormal slow-waves were 6.3±4.8, 9.6±12.6, and 9.0±10.3 for mild blast, mild non-blast, and moderate TBI patients, respectively. No significant group differences were found for the number of gray-matter regions with abnormal slow-waves. The fact that many cortical gray-matter regions showed abnormal MEG slow-waves reveals the diffuse nature of brain injuries in all three TBI groups. No significant hemispheric asymmetry was found for the number of gray-matter regions with abnormal slow-waves in any of the TBI groups.

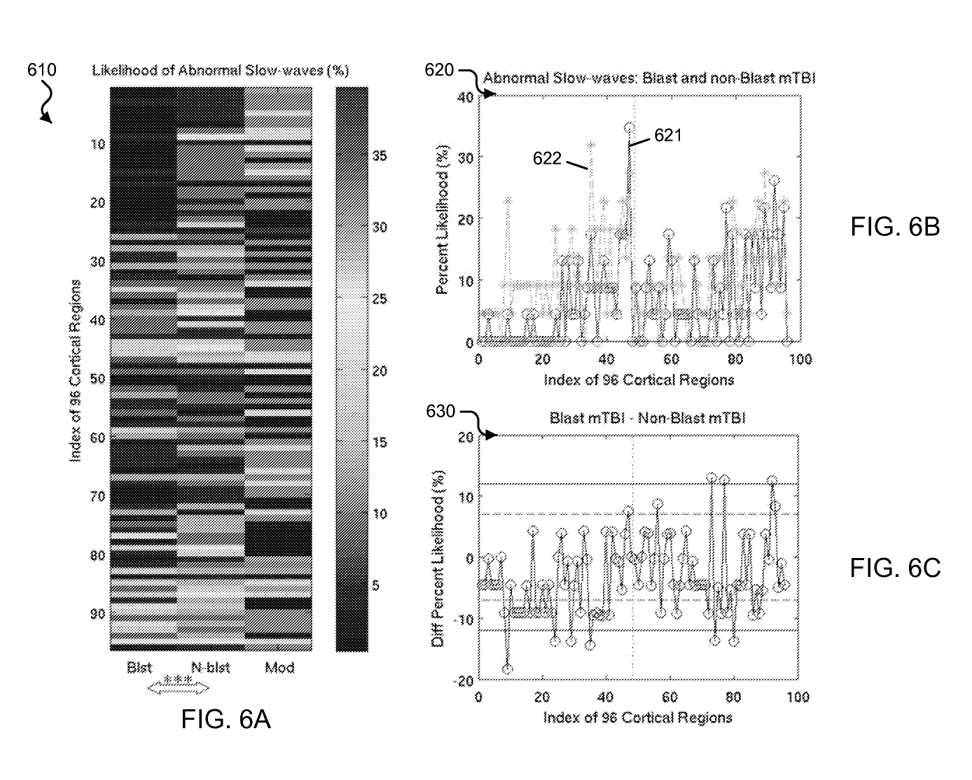

With these three exemplary diagrams in FIG. 5, the data across 96 different gray-matter regions (e.g., regions 1-48 in the left hemisphere, and analogous regions 49-96 in the right) can further be analyzed, and the pattern of neuronal injuries can be estimated, e.g., by calculating the likelihood of slow-wave generation in each cortical gray-matter region within each TBI group. For example, for each row of these diagrams, by summing up across all columns and then dividing the result by the number of patients in each group, the percent likelihood of abnormal slow-wave generation for each cortical gray-matter region was obtained. The result is shown in FIG. 6A, in which the color scale indicates the percent of likelihood for 96 cortical gray-matter regions for the three TBI groups.

FIG. 6A shows a diagram 610 demonstrating the percent likelihood of abnormal slow-wave generation for each cortical gray-matter region in three TBI groups. The two mild TBI groups are highly correlated (double-headed arrow). FIG. 6B shows a data plot 620 demonstrating a different way to plot the percent likelihood of abnormal slow-wave generation for mild blast (blue color, plot 621) and mild non-blast (green color, plot 622) TBI groups. FIG. 6B shows a data plot 630 showing the difference of percent likelihood of abnormal slow-wave generation (blast minus non-blast) showing the non-blast group having fewer regions that were affected by TBI than non-blast group. The exemplary solid (±12%) and exemplary dashed lines (±7%) represent empirical thresholds. The vertical dotted lines in the data plots 620 and 630 divide the regions in the left hemisphere from the ones in the right hemisphere.

For example, the diagram 610 shows a similarity of the pattern between the mild blast TBI (left column) and the mild non-blast TBI (middle column). This exemplary pattern can be seen by the exemplary plots 621 and 622 of the two groups in data plot 620 of FIG. 6B. For example, statistical analysis shows that the pattern of slow-wave generation of the mild blast TBI group is highly and positively correlated with that of the mild non-blast TBI group (r=0.62, p<10⁻¹⁰, df=94), as indicated by the asterisks and double-headed arrow in the diagram 610. In contrast, for example, no significant correlations were found between the mild blast TBI and moderate TBI groups (r=0.07, p=0.48, df=94), or between mild non-blast TBI and moderate TBI groups (r=−0.02, p=0.84, df=94).

Also, for example, despite the highly significant correlation between the patterns of slow-wave generations between mild blast TBI and mild non-blast TBI groups, there are also differences between these two groups. FIG. 6C shows a data plot 630 demonstrating the difference of percent likelihood measure between mild blast versus mild non-blast TBI groups (e.g., the first column minus the second column in the diagram 610 of FIG. 6A, or equivalently, the blue line minus the green line in the data plot 620 of FIG. 6B). The exemplary solid lines in the data plot 630 of FIG. 6C indicate empirical +12% and −12% lines, chosen by visual inspection. The majority of the 96 cortical regions were within these lines, e.g., suggesting that the likelihood of slow-wave generation was similar for these regions. For example, there were only 3 cortical regions that showed higher than 12% in the measure of likelihood difference indicating higher likelihood of slow-wave generation in patients from the mild blast TBI group than the mild non-blast TBI group in these areas. In contrast, for example, twice as many (e.g., 6) cortical areas showed lower than −12% in the measure of likelihood difference which indicates that more patients in the mild non-blast TBI group showed abnormal slow-waves than in the mild blast TBI group in those areas. Also, for example, a similar result was found using the +7% and −7% thresholds in the measure of likelihood (e.g., two dashed lines in the data plot 630 of FIG. 6C). It is noted that only 6 regions that show the blast group greater than non-blast group (e.g., above +7% line) in the exemplary results, whereas 29 regions showed the non-blast greater than blast group (below −7% line).

The exemplary implementation of the MEG source imaging application also included an examination of the relationship between abnormal MEG slow-waves and post-concussive symptoms (PCS) in the exemplary 55 TBI patients. The assessment of PCS in each TBI patient was based on a clinical interview. For example, the symptoms were coded as “1” for existence of symptoms and “0” for absence of symptoms in 28 categories, modified slightly from the Head Injury Symptom Checklist (HISC), e.g., which includes 1) headaches, 2) dizziness, 3) fatigue, 4) memory difficulty, 5) irritability, lack of patience, 6) anxiety, 7) trouble with sleep, 8) hearing difficulties, 9) blurred vision, 10) other visual difficulties, 11) personality changes (e.g., social problems), 12) apathy, 13) lack of spontaneity, 14) affective liability (quick-changing emotions), 15 Depression, 16) Trouble Concentrating, 17) bothered by noise, 18) bothered by light, 19) coordination problems, 20) balance, 21) taste, 22) smell, 23) motor difficulty, 24) difficulty with speech, 25) numbness/tingling, 26) loses temper easily, 27) Sexual Difficulties, 28) Sexual Inappropriateness. For example, the total PCS scores (summing up over all categories) were: 6.4±1.5 for mild blast TBI group, 6.6±3.1 for the mild non-blast TBI group, and 5.4±2.6 for the moderate TBI group. For example, it is noted that no significant group differences were observed among the three different TBI groups. For example, it is noted that none of the healthy control subjects reported any PCS.

For example, to compute the correlation between MEG slow-wave and PCS, the total number of brain regions that generated abnormal slow-waves in each TBI patient were first calculated, e.g., called “N_{slow-wave}_{_{—}}ₛᵤₘ”. Next, for example, the total symptom score was calculated by summing up all 28 PCS categories in each TBI patient, e.g., called “N_{PCS}_{_{—}}ₛᵤₘ”. Then, for example, the correlation between N_{slow-wave}_{_{—}}ₛᵤₘ and N_{PCS}_{_{—}}ₛᵤₘ were computed, and it was found that these two were significantly and positively correlated (r=0.28, p<0.05, df=53). Next, for example, exploratory correlation analyses were performed between the total number of regions that generated MEG slow-waves and the 28 individual PCS categories in these exemplary TBI patients. The exemplary result showed that N_{slow-wave}_{_{—}}ₛᵤₘ significantly and positively correlated with Personality Changes (e.g., social problems) (r=0.35, p<0.01, df=53), Apathy (r=0.35, p<0.01, df=53), and Other Visual Difficulties (r=0.27, p<0.05, df=53). In addition, for example, trends of significant correlations were observed between N_{slow-wave}_{_{—}}ₛᵤₘ with irritability, lack of patience (r=0.22, p=0.09, df=53) and with coordination problems (r=0.23, p=0.08, df=53) in these TBI patients.

The exemplary implementation included a Linear Regression using the Stepwise method, e.g., with MEG slow-wave (N_{slow-wave}_{_{—}}ₛᵤₘ) as the dependent variable and 28 PCS symptoms as the dependent variables. For example, it was found that Personality Changes (e.g., social problems) accounted significantly for the variance of N_{slow-wave}_{_{—}}ₛᵤₘ with R² value of 12% (p<0.01). Furthermore, for example, Other Visual difficulties accounted for additional 9% of the variance, which was also significant (p<0.05). The Stepwise model terminated at this point as the rest of the variables were excluded from analysis due to their p-value being greater than 0.05.

Using the disclosed frequency-domain VESTAL technology in an automated MEG low-frequency source imaging, abnormal delta-waves were found in ˜90% of 45 patients with mild TBI (e.g., 23 with blast and 22 with non-blast causes), and in 100% of 10 patients with moderate TBI (e.g, as shown in FIG. 4 data plot 400). These exemplary positive-finding rates are markedly higher than the ˜9% and 20% rates using the conventional neuroimaging approaches (e.g., CT or MRI) in the same mild and moderate TBI patients, respectively.

The exemplary results also revealed the diffuse nature of the neuronal injuries in TBI patients (e.g., as shown in the diagram 510, 520, and 530 of FIG. 5). For example, the reduced DTI fractional anisotropy in local white-matter fiber tracts led to focal abnormal MEG slow-waves from neighboring gray-matter in mTBI. On the other hand, for example, reduced anisotropy in major white-matter fiber tracts led to multi-focal or distributed patterns of abnormal slow-waves generated from cortical gray-matter areas that can be remote in location but functionally and structurally linked by the injured major/long white-matter fiber tracts.

The exemplary results also revealed the diffuse nature of abnormal MEG slow-wave generation in TBI. For example, unlike abnormal slow-wave generation in patient populations with specific psychiatric and neurological disorders (e.g., such as schizophrenia and Alzheimer's disease, where group-averaging of source locations in space yielded meaningful information about dysfunctional neuronal networks), the loci that showed abnormal slow-wave generations in the exemplary TBI patients tended to be highly variable in location. Hence, for example, group-averaging of MEG slow-wave source locations in space is unlikely to be the most effective way to detect brain injuries. Instead, pattern analyses of the MEG slow-wave generation, such as that introduced in the exemplary implementations of the disclosed technology, can provide more insights about the neuronal injuries in TBI.

The diagnoses used in the exemplary implementations of the VESTAL technology were based on making an objective comparison with a control normative database containing MEG slow-wave source power from 96 cortical regions. The exemplary MEG source imaging analysis was performed by analyzing all the artifact-free epochs from the entire resting-state recording. The exemplary procedure was objective since no human interaction was involved in manually selecting the epochs (e.g., operator independent). For example, the described technique was implemented based on imaging results of slow-waves in source space rather than sensor space. This is substantially different from the conventional approaches, in which an operator with experience selects the sensor-waveform epochs that he/she considers to demonstrate abnormal low-frequency MEG signals.

For example, MEG low-frequency source imaging also can include several advantages and advanced features when implemented using the disclosed frequency-domain VESTAL technology. For example, the described VESTAL techniques can localize neuronal sources with a variety of spatial profiles, e.g., such as focal, multi-focal, dipolar, as well as distributed sources, and a variety of temporal profiles with uncorrelated, partially-correlated, as well as 100% correlated source time-courses. For example, generators of abnormal slow-waves in mild TBI patients can be in one or more of the above spatial-and-temporal profiles, e.g., which is suitable for characterization using the disclosed VESTAL technology. In contrast, for example, conventional MEG slow-wave source analysis uses single-dipole fit which limited its ability to analyze MEG signals with complicated neuronal-source configurations, and which may include some cases of abnormal slow-waves in TBI patients. An additional advantage of the exemplary neuroimaging approach is that it can be implemented with the resting-state MEG recording procedure, e.g., which is spontaneous, requiring almost no effort from TBI patients, and is thus independent of patients' performance and effort.

The disclosed technology was implemented in an exemplary MEG source imaging implementation that examined the diagnostic value of the automated and operator-independent MEG low-frequency (slow-wave) source imaging in mild TBI and moderate TBI. The exemplary results showed that the disclosed VESTAL technology can be used in such implementations, which achieved a positive-finding rate of 90% for the mild TBI group and 100% for the moderate group, e.g., with the threshold chosen so that there were no false-positives in the normal control group. The exemplary results also showed that the patterns of slow-wave generation in mild blast TBI and mild non-blast TBI patients were significantly correlated. The exemplary results also showed significant correlations between the number of cortical regions that generate abnormal slow-waves and the post-concussive symptom scores in TBI patients. The previously described information details the use of mild TBI as a comprehensive example to illustrate the power of MEG source imaging using the disclosed VESTAL technology. For example, the same approach can be used directly to analyze MEG signals (e.g., abnormal slow-wave and source-based functional connectivity) from early Alzheimer's dementia, early multiple sclerosis, autism, schizophrenia, PTSD patients.

In another aspect, the disclosed technology can include a covariance-matrix-based VESTAL technique. The disclosed covariance-matrix-based VESTAL technique can significantly reduce the computational costs of solving the inverse problem using VESTAL, mainly in time-domain data. For example, to analyze MEG data with 30-45 minutes of recording, a conventional time-domain VESTAL may take over 10 hours of computational time. In contrast, the disclosed covariance-matrix VESTAL technology can obtain the MEG source images and associated source time-courses and/or frequency powers in substantially one minute. The disclosed covariance-matrix VESTAL technique can provide complementary source images of the MEG data (e.g., as dominant spatial modes).

An exemplary covariance-matrix VESTAL technique can be complementary to the disclosed frequency-domain VESTAL techniques. For example, if the MEG signal of interest is within a pre-known frequency band (e.g., such as the delta band (e.g., 1-4 Hz), the theta band (e.g., 5-7 Hz), the alpha band (e.g., 8-13 Hz), the beta band (e.g., 15-30 Hz), the gamma band (e.g., 30-100 Hz), or other frequency bands of interest), the frequency-domain VESTAL approach can be a powerful tool for MEG source imaging. On the other hand, for example, if the MEG signal of interest is better represented in time-domain and/or the MEG signals could be distributed across multiple frequency bands, the covariance-matrix-based approach can be valuable, and can provide several advantages. The disclosed technology can also include the combination of the disclosed frequency-domain and covariance-matrix VESTAL techniques to further accelerate the data processing.

An exemplary covariance-matrix-based VESTAL technique can include using the singular value decomposition (SVD) for the G matrix, G=U_{G}S_{G}V_{G}^{T}, the MEG sensor waveform matrix, B(t)=U_{B}S_{B}V_{B} (t)^{T}, and source time-course matrix, Q(t)=U_{Q}S_{Q}V_{Q} (t)^{T} to re-write Eq. 1, and then right-multiply it by V_{B} (t), and left-multiply it by U_{G}^{T} to yield:

U_{G}^{T}U_{B}S_{B}=S_{G}V_{G}^{T}U_{Q}S_{Q} (Eq. 8)

For example, the overall time-information in sensor waveforms and source time-courses are the same (e.g., V_{B} (t)=V_{Q} (t) due to the linear relationship between them. This can be applied in the exemplary technique. For example, in Eq. 8, the time variable can be integrated out, e.g., because V_{B} (t)^{T} V_{B} (t)=V_{Q} (t)^{T} V_{B} (t)=I. The dominant spatial modes in the sensor waveform U_{B} can be easily obtained as eigenvalue decomposition of the covariance matrix R of the sensor-domain data:

$$R = {\frac{1}{N} [ B ( t ) - \overline{B} ] [ B ( t ) - \overline{B} ]}^{T} \qquad ( Eq . 9 )$$

Eq. 8 can be solved using the minimum L1-norm solution with linear programming (e.g., as described in Eqs. 3-5). For example, for each dominant spatial mode in the sensor waveform (e.g., U_{B}), the corresponding MEG imaging in source space (e.g., U_{Q}) can be obtained. Such a solution of the covariance-matrix-based VESTAL can provide a spatial filter that can be run through the MEG time-domain data to obtain the source time-courses with millisecond time-resolution. An exemplary advantage can include only solving for the dominant spatial modes. For example, in a typical MEG recording that lasts 10-20 minutes, which may include 60,000 to 120,000 time samples, the dominant modes of neuronal activities may number less than 40. In this situation, Eq. 8 can be solved in 1-2 minutes, which can drastically reduce the computational cost (e.g., fitting <40 spatial modes vs. fitting 60,000 time samples), which constitutes a huge improvement when analyzing spontaneous MEG signals.

Another exemplary advantage of the disclosed covariance-matrix-based VESTAL technique is that it allows reliable MEG source imaging for evoked MEG signals using fewer averages of the external stimuli (e.g., somatosensory median-nerve stimuli, motor tasks, auditory stimuli, and stimuli for cognitive tasks), e.g., because the covariance-matrix technique is itself an averaging technique (e.g., averaging across time).

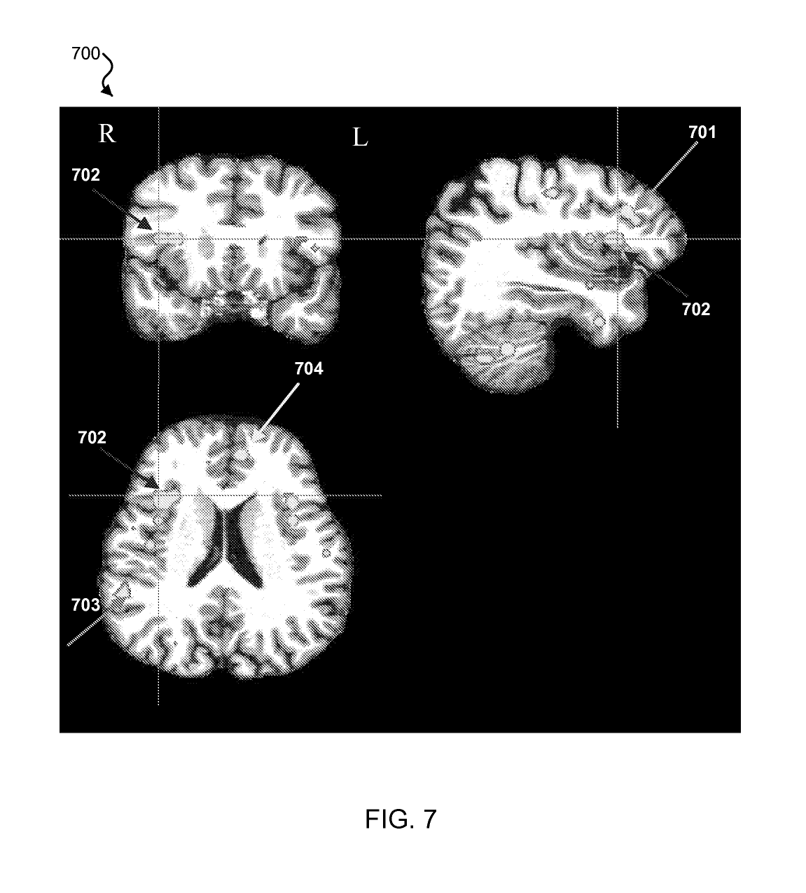

FIG. 7 shows an exemplary covariance-matrix-based VESTAL MEG source imaging diagram 700 that shows the working memory network. The exemplary pink arrow 701 identifies dorsal lateral pre-frontal. The exemplary blue arrows 702 identify ventrolateral pre-frontal. The exemplary green-arrow 703 identifies supra-marginal gyrus. The exemplary yellow arrow 704 identifies anterior cingulate cortex. For example, FIG. 7 shows the result of strong dorsal and ventral-lateral pre-frontal, supramarginal gyrus and anterior cingulate cortex activities (e.g., key areas in the working-memory network) from a working-memory MEG task with only 20 trials of stimuli with poor signal-to-noise ratio (SNR). For example, typically this task can demand 100 trials of stimuli for good SNR. The exemplary covariance-matrix VESTAL not only drastically reduces the computational cost, but also allows good MEG source imaging from low SNR, which can substantially reduce the acquisition time (e.g., by a factor of 5 in the example shown in the diagram 700).

Described are exemplary steps to implement a covariance-matrix-based VESTAL MEG source imaging process, e.g., with high resolution and significantly reduced computational costs, solving the inverse problem using VESTAL, mainly in time-domain data. The process can include a step to compute the MEG sensor covariance matrix based on MEG sensor waveforms in time-domain. For example, the resulting covariance matrix is a square matrix (e.g., number of sensors×number of sensors), with no time-dependence. The process can include a step to obtain the VESTAL source images (e.g., minimum L1-norm inverse solution with ˜10,000 voxels) based on the MEG covariance matrix (e.g., Eqs. 8 and 9). In some examples, the process can include a step to construct a spatial filter based on the MEG covariance matrix VESTAL solution and apply it to the original sensor waveforms, e.g., to obtain the source time-courses.

Implementation of the covariance-matrix-based VESTAL technique can include executing the previously described process 130 and 140 for the exemplary frequency-domain VESTAL technique, in which the exemplary steps are performed in a substantially similar manner. For example, the Fourier components (e.g., the exemplary sensor-space frequency-domain signal Kᵣₑₐₗ and K_{imag}) can be replaced with the covariance-matrix-based spatial modes U_{B}. The process can include a step to apply masks to group the MEG source power from voxels in an image (e.g., ˜10,000 voxels) into a smaller number of brain regions (e.g., as demonstrated in FIGS. 2A-2E), and to develop the MEG covariance-based power vector (e.g., one element for each brain region).

The exemplary covariance-matrix-based VESTAL process can be repeated for a large number of healthy control subjects to develop a healthy control data base for each element/cell of the MEG covariance-based power vector: For example, the process can include a step to calculate the group mean and standard deviation for each cell, e.g., across all the healthy controls. Subsequently, the process can include a step to convert the 1D MEG covariance-based power vector of each healthy control subject into a Z-score vector based on the group mean and standard deviation for each cell. The process can include a step to select the highest Z-value for the entire Z-score vector of each control, and to designate that Z-value to represent that control's maximum Z-score. The process can include a step to choose the highest maximum Z-score of all of the controls, e.g., by setting that value as the threshold to differentiate between normal (e.g., less than or equal to that threshold Z-score) versus abnormally-high delta power (e.g., higher than that threshold Z-score). The exemplary covariance-matrix-based VESTAL process can be repeated for a large number of subjects with neurological or psychiatric disorders (e.g., traumatic brain injury, TBI), and convert the patients' MEG covariance-based power vector into Z-score vectors. For example, regions with Z-scores exceeding the threshold established in the healthy control database can be identified.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, e.g., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non volatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A method for magnetoencephalography (MEG) source imaging, comprising:
selecting signal data associated with one or more frequency bands from a spectrum of the signal data in the frequency domain, the signal data representing magnetic signals emitted by a brain of a subject and detected by a plurality of sensors outside the brain, and the frequency bands including one or more frequencies;
defining locations of sources within the brain that generate the magnetic signals, wherein the number of locations of the sources is selected to be greater than the number of sensors; and
generating a source value of signal power based on the selected signal data corresponding to a respective location of the locations for the one or more frequencies.

2. The method of claim 1, wherein the number of locations of the sources is at least ten times greater than the number of sensors.

3. The method of claim 1, wherein the selecting includes removing other signal data associated with other frequency bands in the generating of the source value of signal power based on the selected signal data.

4. The method of claim 1, further comprising:
converting a data set in time domain format into the spectrum of the signal data in the frequency domain,
wherein the data set includes magnetic signal values detected by the plurality of sensors for a duration of time, each magnetic signal value corresponding to an instance of time of the duration of time and a sensor of the plurality of sensors from which the magnetic signal value was detected.

5. The method of claim 1, further comprising:
applying a mask to generated source values, the mask including regions within the defined locations, wherein each region includes a total source value that is the sum of the source values corresponding to the locations defined within that region.

6. The method of claim 5, wherein the regions represent at least one of cortical, subcortical, or cerebellum gray-matter regions of the brain.

7. The method of claim 1, further comprising:
producing a diagram that represents the source values at the corresponding locations for the one or more frequencies.

8. The method of claim 7, wherein the diagram provides an MEG spatial map of the source values having a resolution of at least one source value per one millimeter volume of the brain.

9. The method of claim 1, wherein the locations correspond to individual voxels within a magnetic resonance imaging (MRI) image of the brain.

10. The method of claim 9, wherein the number of individual voxels is greater or equal to 10,000 voxels.

11. The method of claim 9, further comprising:
producing an image including image features representing the source values at the corresponding locations, wherein the image features are mapped to the corresponding voxels of the MRI image.

12. The method of claim 1, further comprising:
generating a statistical score value for the source values corresponding to each of the locations based on a mean source value and standard deviation value determined from a population of subjects having brains without clinical disorder or injury.

13. The method of claim 12, further comprising:
forming a normative database from a collection of the statistical score values of the subjects having brains without clinical disorder or injury.

14. The method of claim 12, further comprising:
producing a diagram that represents the statistical score values at the locations for the one or more frequencies.

15. The method of claim 13, further comprising:
comparing the source values of the subject to the normative database to detect a brain injury.

16. The method of claim 13, further comprising:
comparing the source values of the subject to the normative database to detect an abnormal neuronal network in the brain.

17. The method of claim 16, wherein:
the abnormal neuronal network corresponds to a neurological or psychiatric disorder including at least one of traumatic brain injury (TBI), stroke, post traumatic stress disorder (PTSD), schizophrenia, Alzheimer's disease/dementia, multiple sclerosis (MS), or autism.

18. The method of claim 1, wherein the magnetic signals are detected through automation without pre-selection of time epochs.

19. The method of claim 1, wherein the magnetic signals are detected without pre-selection of an initial number of locations.

20. The method of claim 1, wherein the source values represent focal and distributed neuronal sources that are localized and resolved with varying degrees of correlations.

21. A method for magnetoencephalography source imaging, comprising:
determining a covariance matrix based on MEG signal data in the time domain, the MEG signal data representing magnetic signals emitted by a brain of a subject and detected by a plurality of sensors outside the brain;
defining locations of sources within the brain that generate the magnetic signals, wherein the number of locations of the sources is selected to be greater than the number of sensors; and
generating a source value of signal power for a respective location of the locations by fitting the covariance matrix.

22. The method of claim 21, wherein the covariance matrix groups neuronal activity into a set of activities.

23. The method of claim 22, wherein the set of activities includes at least 40 neuronal activities.

24. The method of claim 21, further comprising:
wherein the number of locations is at least ten times greater than the number of sensors.

25. The method of claim 21, wherein the locations correspond to individual voxels within an MRI image of the brain.

26. The method of claim 25, further comprising:
producing an image including image features representing the source values at the corresponding locations, wherein the image features are mapped to the corresponding voxels of the MRI image.

27. The method of claim 26, wherein the image includes a resolution of at least one source value per one millimeter volume of the brain.

28. The method of claim 21, further comprising:
generating a statistical score value for the source values corresponding to each of the locations based on a mean source value and standard deviation value determined from a population of subjects having brains without clinical disorder or injury.

29. The method of claim 28, further comprising:
forming a normative database from a collection of the statistical score values of the subjects having brains without clinical disorder or injury.

30. An magnetoencephalography source imaging system, comprising:
an MEG data acquisition system adapted to acquire magnetic signal data emitted by a brain of a subject that are detected by a plurality of sensors outside the brain; and
a data processing unit that receives the magnetic signal data from the MEG data acquisition system, the data processing unit comprising:
a mechanism that converts the acquired magnetic signal data from a time domain format into a spectrum of the magnetic signal data in the frequency domain,
a mechanism that selects signal data associated with one or more frequency bands from a spectrum of the magnetic signal data in the frequency domain, the frequency bands including one or more frequencies, and
a mechanism that generates a source value of signal power based on the selected signal data corresponding to a location within the brain of a source that generates the magnetic signals, wherein the source values are generated for the one or more frequencies.

31. The system of claim 30, wherein the data processing unit further comprises a mechanism that produces a diagram that represents the source values at the corresponding locations, wherein the diagram provides an MEG spatial map of the source values having a resolution of at least one source value per one millimeter volume of the brain.

32. The system of claim 30, further comprising:
a magnetic resonance imaging data acquisition system adapted to acquire magnetic resonance (MR) data from the brain of the subject, the MR data including data voxels,
wherein the locations correspond to individual voxels of the data voxels within an MRI image of the brain.

33. The system of claim 32, wherein the data processing unit further comprises a mechanism that produces an image including image features representing the source values at the corresponding locations, wherein the image features are presented in the data voxels of the MRI image that correspond to the locations within the brain.

34. A method for source imaging, comprising:
selecting signal data associated with one or more frequency bands from a spectrum of the signal data in the frequency domain, the signal data detected by a plurality of sensors oriented about a structure, and the frequency bands including one or more frequencies;
defining locations of sources within the structure, wherein the number of locations of the sources is selected to be greater than the number of sensors; and
generating a source value of signal power based on the selected signal data corresponding to a respective location of the locations for the one or more frequencies.
